(19) United States
(12) Patent Application Publication
(10) Pub. No.: US 2021/0298781 A1
(43) Pub. Date: Sep. 30, 2021

(54) SURGICAL INSTRUMENT

(71) Applicant: Takayama Instrument, Inc., Tokyo (JP)

(72) Inventor: Naoki OTANI, Saitama (JP)

(21) Appl. No.: 17/211,138

(22) Filed: Mar. 24, 2021

(30) Foreign Application Priority Data
Mar. 24, 2020 (JP) 2020-052782

(51) Int. Cl. A61B 17/3201; A61B 17/062; A61B 17/29

(52) U.S. Cl. CPC A61B 17/3201; A61B 17/29; A61B 17/062

(57) ABSTRACT

A surgical instrument includes: a pair of members; and a proximal end portion that connects the pair of members to each other. The pair of members each include a proximal portion, an arm portion, and a raised portion. The proximal portion is a portion at which the proximal end portion is disposed. The arm portion has an end portion on a side that is opposite with respect to the proximal end portion, the opposite end portion being openable and closable. The raised portion has a one-end portion connected to the proximal portion and an other-end portion connected to the arm portion. The raised portion is formed raised so as to be inclined with respect to the proximal portion, and the raised portion has a raised angle of 10 degrees or less.

### RELATED APPLICATIONS

The present application claims priority pursuant to 35 U.S.C. § 119 from Japanese patent application No. 2020-052782, filed on Mar. 24, 2020, the entire disclosure of which is hereby incorporated herein by reference.

### FIELD

The present disclosure relates to a surgical instrument.

### BACKGROUND

Japanese Patent Application Publication No. 2006-288555 and Japanese Patent Application Publication No. 2012-90724 disclose the following instruments. The surgical field, in particular, during an operation in the field of neurosurgery is considerably narrow. Thus, while using an instrument such as an operating microscope, it may be necessary to insert a surgical instrument, such as a scissors blade, through a small gap. As a surgical instrument to be used in such an operation in the field of neurosurgery, a surgical instrument of a bayonet type is known. The bayonet-type surgical instruments each have a portion on the hand side when handheld (hereinafter also referred to as “proximal portion”) and a portion (hereinafter also referred to as “arm portion”) that is to be inserted through a gap toward a surgical field, and the axis of the proximal portion and the axis of the arm portion are displaced from each other because a raised portion is disposed at an intermediate portion in the longitudinal direction. Consequently, it is possible to suppress interference with an instrument, such as an operating microscope.

For example, with a bayonet-type scissors blade, cutting or the like of tissue is performed as a result of a force with which a proximal portion is handheld acting on scissors at the distal end of an arm portion. However, there is sometimes a situation in which the large degree of raising of a raised portion (that is, the axis of the proximal portion and the axis of the arm portion that are largely distanced from each other) results in torsion being generated by the force of handholding, which decreases the nipping force of the scissors. In addition to scissors blades, the same problem occurs to bayonet-type surgical instruments, such as needle holders, forceps and the like, in which the distal end of the arm portion thereof is disposed to be openable and closable.

### BRIEF SUMMARY

An object of the present disclosure is to improve the operability of a bayonet-type surgical instrument.

Some embodiments of the present disclosure is a surgical instrument including: a pair of members; and a proximal end portion that connects the pair of members to each other, the pair of members each including a proximal portion, an arm portion, and a raised portion, the proximal portion being a portion at which the proximal end portion is disposed, the arm portion having an end portion on a side that is opposite with respect to the proximal end portion, the opposite end portion being openable and closable, the raised portion having a one-end portion connected to the proximal portion and an other-end portion connected to the arm portion, the raised portion being formed raised so as to be inclined with respect to the proximal portion, the raised portion having a raised angle of 10 degrees or less.

Some embodiments of the present disclosure is a surgical instrument including: a pair of members; and a proximal end portion that connects the pair of members to each other, the pair of members each including a proximal portion, an arm portion, and a raised portion, the proximal portion being a portion at which the proximal end portion is disposed, the arm portion having an end portion on a side that is opposite with respect to the proximal end portion, the opposite end portion being openable and closable, the raised portion having a one-end portion connected to the proximal portion and an other-end portion connected to the arm portion, the raised portion being formed raised so as to be inclined with respect to the proximal portion, a height between a lower end of the proximal portion and an upper end of the arm portion being 17 mm or less.

Features of the present disclosure other than the above will become clear by reading the description of the present specification with reference to the accompanying drawings.

According to the present disclosure, it is possible to improve the operability of a bayonet-type surgical instrument.

### BRIEF DESCRIPTION OF THE DRAWINGS

FIG. 1 is a perspective view of a surgical instrument 10 according to the present embodiment.

FIG. 2A is a side view of the surgical instrument 10 according to the present embodiment. FIG. 2B is a plan view of the surgical instrument 10 according to the present embodiment.

FIG. 3 illustrates a state in which the surgical instrument 10 according to the present embodiment is handheld.

FIG. 4A is a perspective view of the surgical instrument 10 according to a comparative example. FIG. 4B illustrates a state in which the surgical instrument 10 according to the comparative example is handheld. FIG. 4C is a side view of the surgical instrument 10 according to the comparative example.

FIG. 5A and FIG. 5B illustrate a state in which the surgical instrument 10 according to the comparative example is in use.

FIG. 6 illustrates a state of a flexure test of the surgical instrument 10 according to the present embodiment.

### DETAILED DESCRIPTION

At least the following matters will become clear with the description of this specification and the attached drawings.

A surgical instrument includes: a pair of members; and a proximal end portion that connects the pair of members to each other. The pair of members each include a proximal portion, an arm portion, and a raised portion. The proximal portion is a portion at which the proximal end portion is disposed. The arm portion has an end portion on a side that is opposite with respect to the proximal end portion, the opposite end portion being openable and closable. The raised portion has a one-end portion connected to the proximal portion and an other-end portion connected to the arm portion. The raised portion is formed raised so as to be inclined with respect to the proximal portion, and the raised portion has a raised angle of 10 degrees or less. With such a surgical instrument, it is possible to improve the operability of a bayonet-type surgical instrument.

A surgical instrument includes: a pair of members; and a proximal end portion that connects the pair of members to each other. The pair of members each include a proximal portion, an arm portion, and a raised portion. The proximal portion is a portion at which the proximal end portion is disposed. The arm portion has an end portion on a side that is opposite with respect to the proximal end portion, the opposite end portion being openable and closable. The raised portion has a one-end portion connected to the proximal portion and an other-end portion connected to the arm portion. The raised portion is formed raised so as to be inclined with respect to the proximal portion. A height between a lower end of the proximal portion and an upper end of the arm portion is 17 mm or less. nth such a surgical instrument, it is possible to improve the operability of a bayonet-type surgical instrument.

It is desirable that, when viewed in a longitudinal direction of the proximal portion, a length of the raised portion with respect to a total length of the arm portion and the raised portion is two-fifths or more. Accordingly, it is possible to improve the operability of a bayonet-type surgical instrument.

It is desirable that the surgical instrument is any of a scissors blade, a needle holder, forceps, and a clamp. In this case, such a surgical instrument is particularly advantageous.

### Present Embodiment

### Configuration of Surgical Instrument in Present Embodiment

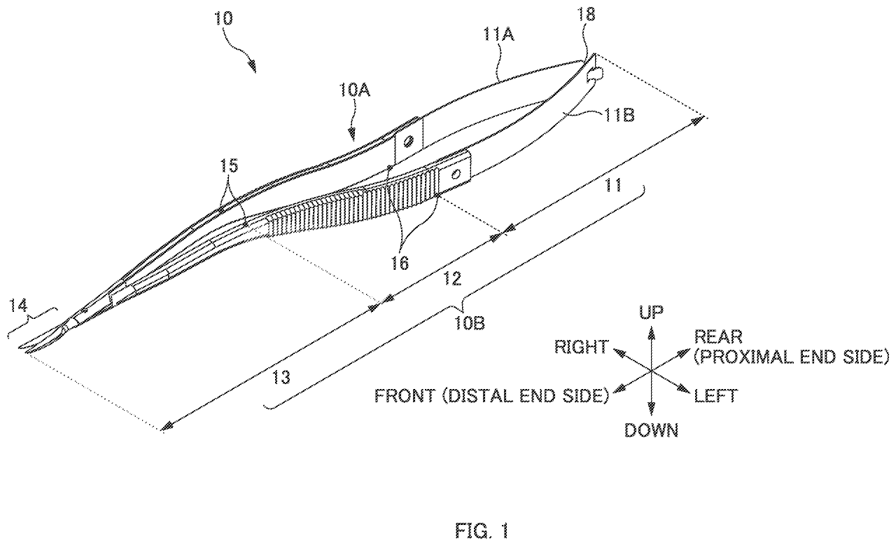

FIG. 1 is a perspective view of the surgical instrument 10 according to the present embodiment.

Hereinafter, the configuration and the action of the surgical instrument 10 are sometimes described on the basis of the directions indicated in FIG. 1. That is, the longitudinal direction of a proximal portion 11 of the surgical instrument 10 (a right member 10A or a left member 10B) is referred to as “front-rear direction”, the side of an arm portion 13 as viewed from a raised portion 12 is referred to as “front”, and the side of the proximal portion 11 as viewed from the raised portion 12 is referred to as “rear”. Note that the front side and the rear side are also referred to as “distal end side” and “proximal end side”, respectively. The direction in which the right member 10A and the left member 10B of the surgical instrument 10 are disposed side by side is referred to as “left-right direction”. The direction in which the “front-rear direction” and the “left-right direction” are orthogonal to each other is referred to as “up-down direction”.

The surgical instrument 10 is a medical instrument for use in an operation on a human body. In the present embodiment, the surgical instrument 10 is for use in an operation in the field of neurosurgery. Note that, in addition to the field of neurosurgery, the surgical instrument 10 may also be used in operations in the fields of plastic surgery, orthopedic surgery, transplant surgery, and the like. In the present embodiment, the distal end side of the surgical instrument 10 is disposed to be openable and closable. The surgical instrument 10 is, for example, a scissors blade, a needle holder, forceps, or a clamp. Note that the surgical instrument 10 may be an instrument other than a scissors blade, needle holders, forceps, and clamps. Hereinafter, the surgical instrument 10 as a scissors blade will be described.

In the present embodiment, the surgical instrument 10 is formed of stainless steel. Note that the surgical instrument 10 may be formed of a material other than stainless steel, such as titanium, an alloy thereof, and the like. Different materials may be used for each of the portions of the surgical instrument 10 (for example, the proximal portion 11, the raised portion 12, and the arm portion 13, which will be described later).

As illustrated in FIG. 1, in the present embodiment, a pair of members (the right member 10A and the left member 10B) are disposed to face each other with the distal end sides (distal end portions 14) thereof being disposed to be openable and closable. On the rear side of the distal end portions 14, the pair of members are disposed to be held by fingertips and to be approachable to and separable from each other.

The surgical instrument 10 includes the right member 10A, the left member 10B, and a proximal end portion 18.

The right member 10A and the left member 10B are a pair of members that constitute the surgical instrument 10. Hereinafter, the right member 10A and the left member 10B are also referred to as “the pair of members”.

The pair of members each include the proximal portion 11, the raised portion 12, and the arm portion 13.

The proximal portion 11 is a portion at which the proximal end portion 18 is disposed. The proximal portion 11 of the right member 10A includes a right extending portion 11A, and the proximal portion 11 of the left member 10B includes a left extending portion 11B. Each of the right extending portion 11A and the left extending portion 11B has a cantilever shape extending from the proximal end portion 18 toward the front side. Consequently, the proximal portion 11 is disposed to be elastically deformable in a direction of opening and closing in the left-right direction. When the pair of members are gripped by fingertips, the proximal portion 11 (the right extending portion 11A and the left extending portion 11B) is deformed in a closing direction. When the deformed proximal portion 11 (the right extending portion 11A and the left extending portion 11B) is restored in an opening direction, the pair of members return to their respective original positions. Due to the proximal portion 11 being disposed to be elastically deformable in the direction of opening and closing in the left-right direction, the opening and closing action of the distal end portions 14 is easily performed.

The raised portion 12 is a portion that connects the proximal portion 11 and the arm portion 13 to each other. The rear end portion of the raised portion 12 is connected to the proximal portion 11, and the front end portion of the raised portion 12 is connected to the arm portion 13. The raised portion 12 is formed being raised so as to be inclined with respect to the proximal portion 11. Consequently, the center axis of the arm portion 13 connected to the front end portion of the raised portion 12 is displaced with respect to the center axis of the proximal portion 11. The surgical instrument 10 in which the center axis of the arm portion 13 is thus displaced with respect to the center axis of the proximal portion 11 is called a surgical instrument of a “bayonet type”. A surgical instrument that does not include the raised portion 12 and in which the center axis of the proximal portion 11 and the center axis of the arm portion 13 are the same is called a straight-type surgical instrument 10. The bayonet-type surgical instrument can suppress interference with instruments such as an operating microscope and the like, in contrast to the straight-type surgical instrument.

The arm portion 13 is a portion that is to be inserted toward a surgical field. The arm portion 13 has the distal end portion 14 at its distal end (an end portion on a side that is opposite with respect to the proximal end portion 18). The distal end portions 14 are disposed to be openable and closable. In the present embodiment, scissors (scissors blade) are disposed as the distal end portions 14. Note that the distal end portions 14 may be an instrument such as a needle holder, forceps, a clamp or the like, that is disposed to be openable and closable. The length of the arm portion 13 is also referred to as an effective length.

The proximal end portion 18 is a portion that connects the pair of members to each other. The proximal end portion 18 is disposed at a rear end portion of the surgical instrument 10. As illustrated in FIG. 1, the proximal end portion 18 is disposed as an intersection portion between the right member 10A and the left member 10B.

### Details of Raised Portion in Present Embodiment

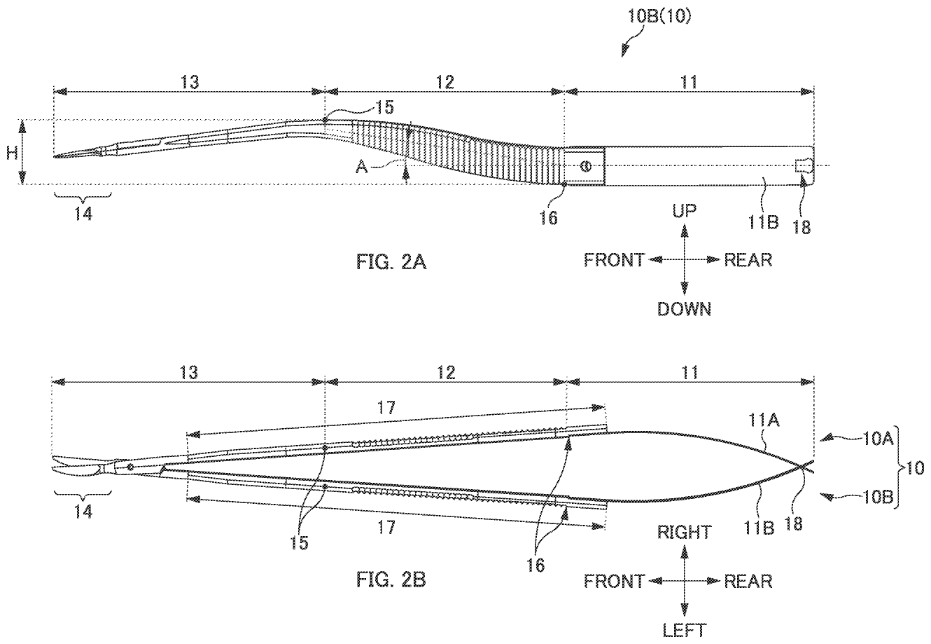

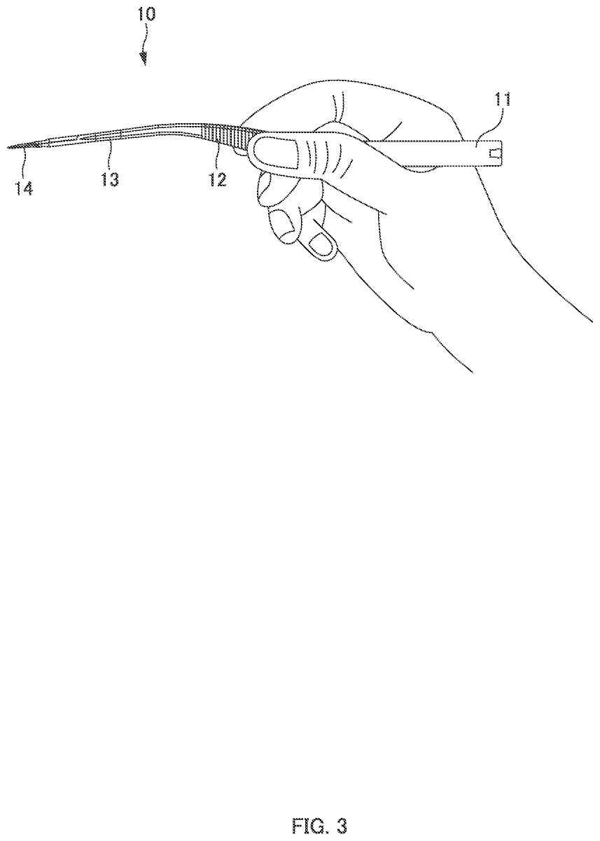

FIG. 2A is a side view of the surgical instrument 10 according to the present embodiment. FIG. 2B is a plan view of the surgical instrument 10 according to the present embodiment. FIG. 3 illustrates a state in which the surgical instrument 10 according to the present embodiment is handheld.

As illustrated in FIG. 2A, in the present embodiment, the raised portion 12 is formed being raised so as to be inclined with respect to the proximal portion 11. The raised portion 12 includes a raise start portion 16 and a top portion 15. The raise start portion 16 is a portion at which the raised portion 12 starts to be raised, in the side view of the surgical instrument 10 illustrated in FIG. 2A. The top portion 15 is a portion at which the raised portion 12 is raised most (positioned on the upper side), in side view of the surgical instrument 10 illustrated in FIG. 2A.

In the present embodiment, the raised portion 12 is formed being raised smoothly. Specifically, the raised portion 12 is formed such that a raised angle A thereof illustrated in FIG. 2A is 10 degrees or less. In the present embodiment, the raised angle A is the angle of the following line with respect to the center axis of the proximal portion 11: a line connecting a point on the center axis of the raised portion 12 at the position of the raise start portion 16 and a point on the center axis of the raised portion 12 at the position of the top portion 15.

As illustrated in FIG. 2A, the raised portion 12 is formed such that a height H between the lower end (the raise start portion 16) of the proximal portion 11 and the upper end (top portion 15) of the arm portion 13 is 17 mm or less. This makes the raised portion 12 is raised smoothly.

As illustrated in FIG. 2A, the raised portion 12 is also formed such that the length thereof with respect to the total length of the arm portion 13 and the raised portion 12 is two-fifths or more. This makes the raised portion 12 is raised smoothly.

As illustrated in FIG. 3, the surgical instrument 10 according to the present embodiment is normally held in a holding manner that is called a pen-holder method. In this holding manner, with the tip of the thumb and the tips of the index finger and the middle finger being placed on the surfaces of the pair of arms, the lower portions of the pair of arms on the proximal end side are supported by a portion near the base of the index finger. In such a holding manner, the pair of arms are in contact at four points.

### SURGICAL INSTRUMENT IN COMPARATIVE EXAMPLE

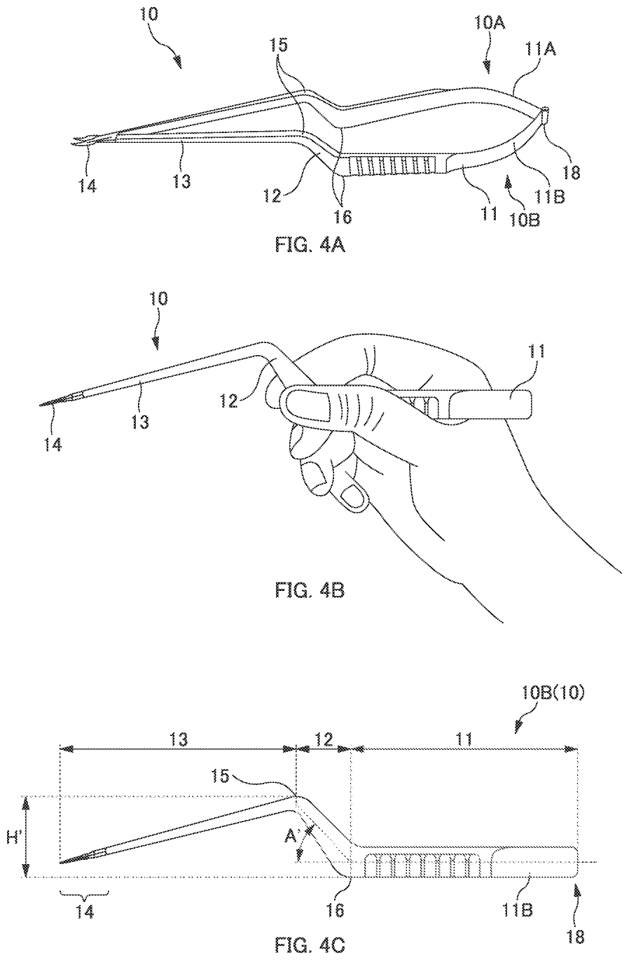

FIG. 4A is a perspective view of the surgical instrument 10 according to a comparative example. FIG. 4B illustrates a state in which the surgical instrument 10 according to the comparative example is handheld. FIG. 4C is a side view of the surgical instrument 10 according to the comparative example.

The surgical instrument 10 according to the comparative example is formed such that the raising of the raised portion 12 is steep in contrast to the surgical instrument 10 according to the present embodiment. In other words, the surgical instrument 10 according to the comparative example is formed such that a raised angle A′ of the raised portion 12 is significantly larger than 10 degrees in contrast to the surgical instrument 10 according to the present embodiment. The surgical instrument 10 according to the comparative example is also formed such that a height H′ between the lower end of the proximal portion 11 and the upper end of the arm portion 13 is significantly larger than 17 mm in contrast to the surgical instrument 10 according to the present embodiment. Moreover, the surgical instrument 10 according to the comparative example is also formed such that the length of the raised portion 12 with respect to the total length of the arm portion 13 and the raised portion 12 is significantly smaller than two-fifths in contrast to the surgical instrument 10 according to the present embodiment. In the surgical instrument 10 according to the comparative example, due to the raising of the raised portion 12 being formed to be steep, the center axis of the proximal portion 11 and the center axis of the arm portion 13 are largely distanced from each other.

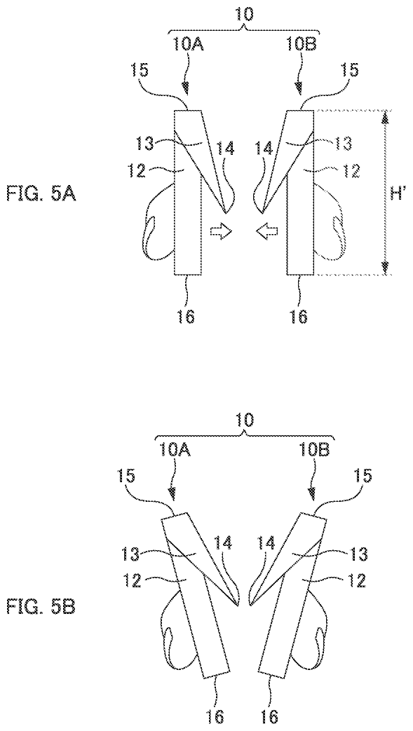

FIG. 5A and FIG. 5B illustrate a state of the surgical instrument 10 according to the comparative example is in use. FIG. 5A and FIG. 5B each illustrate forceps (tweezers) as the distal end portions 14, for ease of description.

As illustrated in FIG. 5A and FIG. 5B, in the surgical instrument 10 according to the comparative example, due to the raising of the raised portion 12 being formed to be steep, torsion is generated by a force of handholding the surgical instrument 10. In other words, as illustrated in FIG. 5A, when the pair of members are held by fingertips so as to cause the pair of the members to approach each other, the degree of approach in the vicinity of the raise start portion 16 of the raised portion 12 is large, and the degree of approach in the vicinity of the top portion 15 of the raised portion 12 is small, as illustrated in FIG. 5B. Therefore, even when the pair of members are held by fingertips such that the pair of members approach each other, torsion may be generated in the pair of members, decreasing the degree of approach of the distal end portions 14. Thus, in the surgical instrument 10 according to the comparative example, there is a risk of decreasing of the nipping force of the distal end portions 14.

However, in the surgical instrument 10 according to the present embodiment, that the raising of the raised portion 12 is formed to be smooth makes it possible to suppress generation of torsion in the pair of members. Therefore, in the surgical instrument 10 according to the present embodiment, it is possible to suppress decreasing of the nipping force of the distal end portions 14. In other words, the action of a cutting operation of a hand is easily transferred to the distal end portions 14 as a shearing force.

As illustrated in FIG. 4B, in the surgical instrument 10 according to the comparative example, due to the raising of the raised portion 12 being steep, a holding position by fingertips is limited to the part of the proximal portion 11. This is because, when the part of the raised portion 12 is held with fingertips, the visibility of a surgical field is considerably degraded due to obstruction by the fingertips. However, in the surgical instrument 10 according to the present embodiment, due to the raising of the raised portion 12 being smooth, the portion to be held by fingertips is finely adjustable. That is, the holding position by fingertips is not limited to only the part of the proximal portion 11 and is displaceable toward the raised portion 12. Even when the holding position by fingertips is finely adjusted, the visibility of a surgical field is favorable. Further, due to the holding position by fingertips being finely adjustable, it is also possible to improve the stability of the distal end portions 14.

In some cases, during an operation in which the surgical instrument 10 is used, an operation of rotating about the longitudinal direction (front-rear direction) of the proximal portion 11 is performed. Even in this case, the surgical instrument 10 of the present embodiment makes it easier for fingertips to displace the holding position toward the raised portion 12 and holding the surgical instrument 10, making it possible to easily perform such a rotating operation. That is, during a rotating operation, rotation from an elbow is not required, and a fine rotating operation is easily performed with fingertips.

### Flexure Test

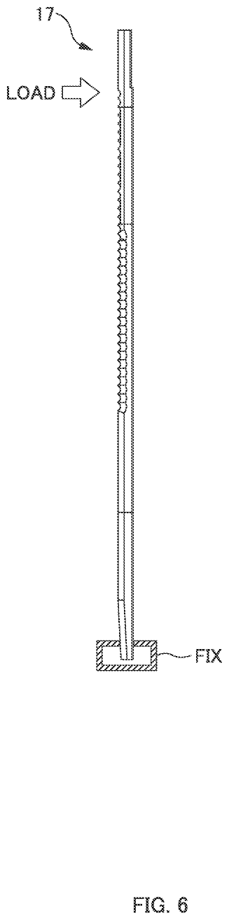

FIG. 6 illustrates a state of a flexure test of the surgical instrument 10 according to the present embodiment.

In order to confirm that torsion is suppressed by the holding force of fingertips in the surgical instrument 10 according to the present embodiment, the degree of flexure when a load is applied was tested. In the flexure test, with the distal end side of a trunk portion 17 illustrated in FIG. 2B being fixed, a load of 1 N in the left-right direction was applied to the proximal end side thereof. At this time, in the surgical instrument 10 according to the present embodiment, the amount of movement of a proximal-end-side end portion of the trunk portion 17 was 0.4586 mm. In the surgical instrument 10 according to the comparative example, the amount of movement of the proximal-end-side end portion of the trunk portion 17 was 1.403 mm. That is, the flexure of the trunk portion 17 when the same load (1 N) was applied was significantly smaller in the surgical instrument 10 according to the present embodiment than in the surgical instrument 10 according to the comparative example. This means that, in the surgical instrument 10 according to the present embodiment, the action of a cutting operation of a hand is easily transferred to the distal end portions 14 as a shearing force in contrast to the surgical instrument 10 according to the comparative example.

### Other Embodiments

Although the embodiment of the present disclosure has been described hereinabove, the above embodiment of the present disclosure is simply to facilitate understanding of the present disclosure and are not in any way to be construed as limiting the present disclosure. The present disclosure may variously be changed or altered without departing from its gist and encompass equivalents thereof.

1. A surgical instrument comprising:
a pair of members; and
a proximal end portion that connects the pair of members to each other,
the pair of members each including a proximal portion, an arm portion, and a raised portion,
the proximal portion being a portion at which the proximal end portion is disposed,
the arm portion having an end portion on a side that is opposite with respect to the proximal end portion,
the opposite end portion being openable and closable,
the raised portion having a one-end portion connected to the proximal portion and
an other-end portion connected to the arm portion,
the raised portion being formed raised so as to be inclined with respect to the proximal portion,
the raised portion having a raised angle of 10 degrees or less.

2. A surgical instrument comprising:
a pair of members; and
a proximal end portion that connects the pair of members to each other,
the pair of members each including a proximal portion, an arm portion, and a raised portion,
the proximal portion being a portion at which the proximal end portion is disposed;
the arm portion having an end portion on a side that is opposite with respect to the proximal end portion,
the opposite end portion being openable and closable,
the raised portion having
a one-end portion connected to the proximal portion and
an other-end portion connected to the arm portion,
the raised portion being formed raised so as to be inclined with respect to the proximal portion,
a height between a lower end of the proximal portion and an upper end of the arm portion being 17 mm or less.

3. The surgical instrument according to claim 1, wherein
when viewed in a longitudinal direction of the proximal portion,
a length of the raised portion with respect to a total length of the arm portion and the raised portion is two-fifths or more.

4. The surgical instrument according to claim 2, wherein
when viewed in a longitudinal direction of the proximal portion,
a length of the raised portion with respect to a total length of the arm portion and the raised portion is two-fifths or more.

5. The surgical instrument according to claim 1, wherein
the surgical instrument is any of a scissors blade, a needle holder, forceps, and a clamp.

6. The surgical instrument according to claim 2, wherein
the surgical instrument is any of a scissors blade, a needle holder, forceps, and a clamp.

7. The surgical instrument according to claim 3, wherein
the surgical instrument is any of a scissors blade, a needle holder, forceps, and a clamp.
